(19) United States
(12) Patent Application Publication
(10) Pub. No.: US 2021/0186517 A1
(43) Pub. Date: Jun. 24, 2021

(54) FLOW OCCLUSION DEVICE HAVING CIRCUMFERENTIAL FLUSHING

(71) Applicant: Revamp Medical Ltd.

(72) Inventors: Michael G. TAL, Savyon (IL); Yael SHOHAT, Herzliya (IL); Boaz SCHWARZ, Bat Yam (IL)

(21) Appl. No.: 16/642,812

(22) PCT Filed: Apr. 15, 2018
(86) PCT No.: PCT/IB2018/052608

§ 371 Date: Feb. 27, 2020

Related U.S. Application Data
(60) Provisional application No. 62/488,916, filed on Apr. 24, 2017.

(51) Int. Cl. A61B 17/12

(52) U.S. Cl. CPC A61B 17/12136; A61B 17/12109

(57) ABSTRACT

A blood occlusion device (10) includes a catheter shaft (12), and an expandable occlusion member (14) assembled with the catheter shaft (12). The expandable occlusion member (14) includes interconnecting struts (16). Distal ends of at least some of the struts (16) have a foldable protrusion (20) connected thereto by a hinge member (22). The foldable protrusions (20) pivot about at least some of the struts (16) in one or more directions. One or more connecting links (26) are connected to the foldable protrusions (20). Manipulation of the connecting links (26) and the foldable protrusions (20) modifies occlusion ability of the foldable protrusions (20). A covering (24) covers the foldable protrusions (20). The covering (24) is impervious to blood flow. An outer contour of the covering (24) includes one or more non-smooth portions (36).

### FIELD OF THE INVENTION

The present invention generally relates to apparatus and methods for altering blood flow or for altering or affecting preload and afterload in the cardiovascular system, such as to treat different conditions, such as but not limited to, venous hypertension or pulmonary edema in acute CHF (chronic heart failure) patients, and particularly to blood flow occlusion devices with structure to promote flow to flush around the occlusion device.

### BACKGROUND OF THE INVENTION

PCT patent application PCT/IB2016/055763 describes a blood flow reducing assembly or flow occlusion device for altering or affecting blood flow or for altering or affecting preload and afterload in the cardiovascular system, such as to treat different conditions, such as but not limited to, pulmonary edema in acute CHF (chronic heart failure) patients or venous hypertension and other conditions.

The blood flow reducing assembly includes a self-expandable element located at a distal end of an indwelling catheter. The self-expandable element has a distal end with a plurality of circumferentially placed, inwardly folding elements with hinge members, which allow the foldable elements to bend inwards. The foldable elements and/or self-expandable elements are coated or covered with a membrane or other covering, which is impervious to blood flow. The degree of closure of the device is controlled by an operator using a handle located at the proximal end of the catheter.

The covering of the blood flow reducing assembly contacts the inner vascular wall and this contact can pose a problem, as is now explained.

Prolonged contact between a foreign body and the vascular wall may cause endothelial damage and even breakdown of the endothelium. The endothelium plays a crucial role in providing the proper hemostatic balance. The function of endothelial cells far exceeds that of providing a non-thrombogenic inner layer of the vascular wall that helps to maintain blood fluidity. Under physiological conditions, endothelial cells prevent thrombosis by means of different anticoagulant and antiplatelet mechanisms. These cells are involved in all main hemostatic pathways triggered upon vascular injury and limit clot formation to the areas where hemostasis is needed to restore vascular integrity. Breakdown of this complex balance between pro- and anticoagulant systems because of genetic or acquired disturbances may result in bleeding or thrombosis. Endothelial heterogeneity assures adequate homeostasis in the different organs and parts of the vascular tree. The local environment elicits heterogeneous endothelial cell phenotypes determined by local needs. This heterogeneity also explains the diverse pathological responses to a disturbed vascular integrity. Localized manifestation of thrombosis in spite of disturbance of systemic pro-coagulant systems depends on vascular bed-specific properties. Endothelial dysfunction not only precedes atherogenesis but may also predispose to arterial thrombosis. The potential role of the endothelium in venous thrombosis with and without overt vessel wall injury has been discussed. The vast majority of endothelial cells are located in the microvessels. Therefore, it is no surprise that endothelial cells play a key role in microcirculatory diseases such as thrombotic microangiopathies and diffuse intravascular coagulation. Microcirculatory endothelial cell activation is an important feature in all thrombotic microangiopathies. In diffuse intravascular coagulation, the endothelium is the interface between inflammation and inappropriate activation of the coagulation system.

### SUMMARY OF THE INVENTION

The present invention seeks to provide a blood flow occlusion device with structure to promote blood flow to flush around the occlusion device and thus promote endothelial heterogeneity, assuring adequate homeostasis and preventing endothelial dysfunction.

The invention provides a delicate balance between flushing and obstruction. On the one hand, the device of the invention is a flow occlusion device which is designed to obstruct blood flow to treat different conditions, such as but not limited to, pulmonary edema in acute CHF (chronic heart failure) patients or venous hypertension and other conditions. On the other hand, the device of the invention has a circumferential flushing mechanism to promote endothelial heterogeneity. Too much flushing detracts from the efficacy of the obstructing element by allowing leakage around the occluding covering. The invention structures the occluding covering to prevent over-flushing and maintain a delicate balance between flushing and obstruction. In one embodiment, the occluding cover structure is a thin flow resisting membrane which has an uneven or saw-tooth like edge which decreases the contact area between the membrane and the blood vessel thereby allowing minimal but consistent blood flow around the occluding membrane and the vessel wall.

The flow occlusion device with its thin membrane covering may experience platelet adhesion which can lead to thrombus formations on the device. These formations may dislodge from the device and lead to serious clinical results. In order to prevent such risks, the expandable device and the thin blood obstructing membrane may be coated with materials which greatly reduce the risk of platelet adhesion.

There is provided in accordance with a non-limiting embodiment of the invention a blood occlusion device including a catheter shaft, an expandable occlusion member assembled with the catheter shaft, the expandable occlusion member including interconnecting struts, wherein distal ends of at least some of the struts have a foldable protrusion connected thereto by a hinge member, such that the foldable protrusions pivot about the at least some of the struts in one or more directions, one or more connecting links connected to the foldable protrusions, wherein manipulation of the one or more connecting links and the foldable protrusions modifies occlusion ability of the foldable protrusions, and a covering that covers the foldable protrusions, the covering being impervious to blood flow, wherein an outer contour of the covering includes one or more non-smooth portions.

In accordance with an embodiment of the invention a method is provided for blood occlusion including installing the device of the invention in a blood vessel, and permitting blood to flow along the one or more non-smooth portions between the outer contour of the covering and an inner wall of the blood vessel.

### BRIEF DESCRIPTION OF THE DRAWINGS

The present invention will be understood and appreciated more fully from the following detailed description, taken in conjunction with the drawings in which:

FIGS. 1A and 1B are simplified pictorial illustrations of a blood flow reducing assembly (also called a blood occlusion device), constructed and operative in accordance with a non-limiting embodiment of the invention.

FIG. 2 is a simplified pictorial illustration of the blood occlusion device in-situ within a blood vessel. Flow lines show the flow through the center of the device (partially occluding blood flow in the vessel) and around the circumference of the device between the inner wall of the vessel and the device.

FIG. 3 is a simplified pictorial illustration of the blood occlusion device within a blood vessel allowing some blood flow around the device when in the partially or fully occluded state.

### DETAILED DESCRIPTION OF EMBODIMENTS

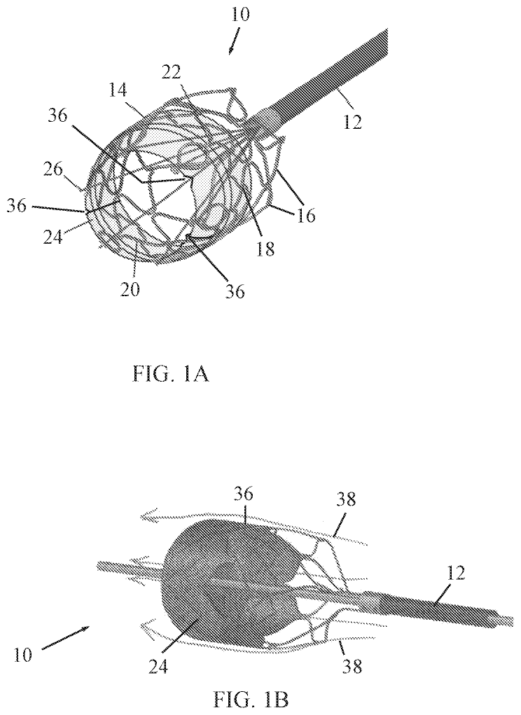

Reference is now made to FIG. 1A, which illustrates a blood occlusion device 10, constructed and operative in accordance with a non-limiting embodiment of the invention.

Device 10 includes a shaft, such as a flexible catheter shaft 12, and an expandable occlusion member 14 assembled with shaft 12. The occlusion member 14 may be initially disposed in shaft 12 and deployed out of shaft 12 such as by pushing occlusion member 14 out of shaft 12. Alternatively, occlusion member 14 may be mounted at the distal end of shaft 12 (not inside shaft 12). The expandable occlusion member 14 may be self-expanding (e.g., constructed of a shape memory material, such as but not limited to, nitinol) or expandable by mechanical means (e.g., wires that push/pull expandable elements) or expandable by fluid means (e.g., hydraulic or pneumatic inflation/deflation of flexible members, such as but not limited to, balloons).

The expandable occlusion member 14 may expand radially (and/or axially) and conform to the shape of the body lumen (e.g., blood vessel) in which it is deployed. The expandable occlusion member 14 may be generally cylindrical in shape (although other shapes are within the scope of the invention). The expanded size of occlusion member 14 may be greater than the internal perimeter of the body lumen, so that occlusion member 14 may be used to remodel the shape of the body lumen.

In this embodiment, the occlusion member 14 is constructed of interconnecting struts 16, such as wires or other slender elements, which may be bent or otherwise formed into loops that are interconnected at wire folds 18. This structure of interconnecting struts 16 can be easily compressed and subsequently expanded to a predetermined shape.

The expandable occlusion member 14 may include one or more foldable protrusion 20 (which may be formed as loops). The foldable protrusions 20 may be connected to at least some (or all) of the struts 16. In the illustrated embodiment, foldable protrusion 20 is connected to the distal end of the strut 16 by a hinge member 22, which allows the foldable protrusion 20 to pivot about the distal end of the strut 16 in one or more directions. Hinge members 22 are circumferentially distributed about the distal ends of some or all of the struts 16. The foldable protrusions 20 can extend axially from hinge members 22 in a fully open position or can fold inwards towards each other in a partially or fully closed position.

A covering 24 is provided at the distal end of assembly 10. Covering 24 may cover the foldable protrusions 20 and may also cover part of the distal ends of the struts 16 and part of the hinge members 22 or other parts of occlusion member 14. Covering 24 may be a membrane which is impervious to blood flow. One or more connecting links 26, such as wires or threads and the like, may be connected to (e.g., the distal end of) each of the foldable protrusions 20. The connecting links 26 extend through the axial length of the catheter shaft 12 to a handle (not shown) located at a proximal end of shaft 12. The handle may be used to pull or otherwise manipulate connecting links 26, thereby pulling the foldable protrusions 20 inwards in a radial direction, effectively creating a resistance to the flow in the body lumen. In other words, movement of the foldable protrusions 20 closer to one another creates or increases occlusion of the flow in the body lumen.

In one embodiment, the outer contour of the occluding covering 24 includes one or more non-smooth portions 36, such as folds, bulges, uneven edges, sinusoidal edges, wavy edges, saw-tooth edges and the like, which decrease the contact area between covering 24 and the inner blood vessel wall. The non-smooth portions 36 create pathways for minimal but consistent blood flow around covering 24 between covering 24 and the vessel wall.

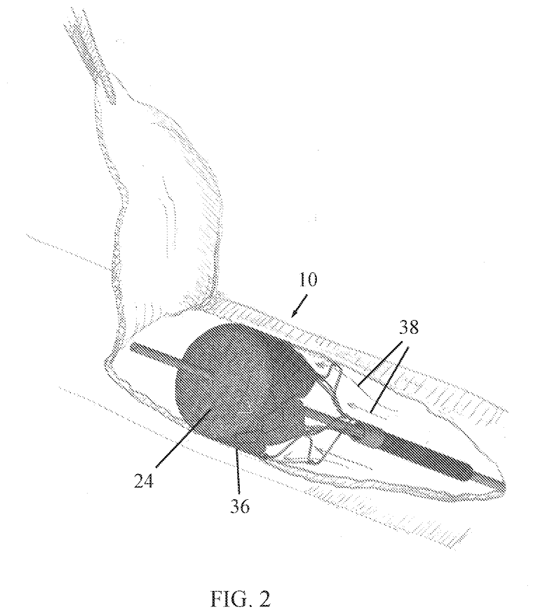

FIG. 1B illustrates flow lines 38 around covering 24 of blood occlusion device 10. FIG. 2 illustrates blood occlusion device 10 in-situ within a blood vessel. Flow lines 38 show the flow through the center of the device (with covering 24 partially occluding blood flow in the vessel; the foldable protrusions 20 may be moved inwards to create a space which is left open for flow through the center of the covering 24 of the device 10) and around the circumference of device 10 between the inner wall of the vessel and covering 24.

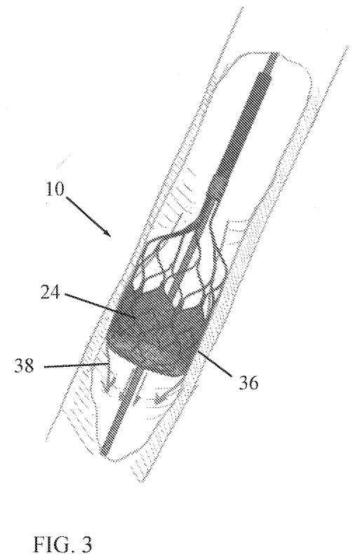

FIG. 3 illustrates blood occlusion device 10 within a blood vessel allowing some blood flow around covering 24 when in the partially or fully occluded state.

In order to prevent thrombus formations on device 10, covering 24 may be coated with a non-thrombogenic material which greatly reduce the risk of platelet adhesion, such as but not limited to, heparin.

What is claimed is:

1. A blood occlusion device (10) comprising:
a catheter shaft (12);
an expandable occlusion member (14) assembled with said catheter shaft (12), said expandable occlusion member (14) comprising interconnecting struts (16), wherein distal ends of at least some of said struts (16) have a foldable protrusion (20) connected thereto by a hinge member (22), such that said foldable protrusions (20) pivot about said at least some of said struts (16) in one or more directions;
one or more connecting links (26) connected to said foldable protrusions (20), wherein manipulation of said one or more connecting links (26) and said foldable protrusions (20) modifies occlusion ability of said foldable protrusions (20); and
a covering (24) that covers said foldable protrusions (20), said covering (24) being impervious to blood flow;
wherein an outer contour of said covering (24) comprises one or more non-smooth portions (36).

2. The device (10) according to claim 1, wherein said one or more non-smooth portions (36) comprises one or more folds.

3. The device (10) according to claim 1, wherein said one or more non-smooth portions (36) comprises one or more bulges.

4. The device (10) according to claim 1, wherein said one or more non-smooth portions (36) comprises one or more uneven edges.

5. The device (10) according to claim 1, wherein said covering (24) also covers part of the distal ends of said struts (16) and part of said hinge members (22).

6. The device (10) according to claim 1, wherein when said foldable protrusions (20) are moved inwards, a space is left open in said covering (24).
